(19) United States
(12) Patent Application Publication
(10) Pub. No.: US 2025/0332002 A1
(43) Pub. Date: Oct. 30, 2025

(54) CLASP MECHANISM

(71) Applicant: RCM ENTERPRISE LLC, Olympia, WA (US)

(72) Inventors: Rose Wood GILRAEN, Olympia, WA (US); Rachel Adsit LOWING, Olympia, WA (US); Bradley Arthur CRITTENDEN, Olympia, WA (US)

(21) Appl. No.: 19/192,693

(22) Filed: Apr. 29, 2025

Related U.S. Application Data
(60) Provisional application No. 63/640,024, filed on Apr. 29, 2024.

(51) Int. Cl. A61F 2/58; A61F 2/78

(52) U.S. Cl. CPC A61F 2/586; A61F 2002/7862

(57) ABSTRACT

A clasp mechanism includes a clasp for securing a first strap segment to a second strap segment. The clasp includes a cinch and a bracket. The clasp features a tensile, high-load catch feature for securing the clasp mechanism during wear and a compressive, low-load hold feature that provides toggle force resistance via strap material compression.

### FIELD OF DISCLOSURE

The present disclosure relates to a clasp mechanism for attaching a cinch to a bracket, which may be used in orthopedic and prosthetic devices, as well as any other device requiring a clasp mechanism.

### BACKGROUND

Finger amputations are the most common type of upper limb amputations, primarily affecting single digits. When a person loses finger mobility, functionality, or any part of their physical finger, the result is impaired hand performance. Having an amputated or minimally functioning finger (e.g., due to nerve damage, excessive scar tissue, neurological disorders, or other dysfunctions of the bones or muscles) restricts the individual from performing some of the most basic tasks. For instance, typing on a computer keyboard or dialing a telephone becomes significantly more challenging with one or more dysfunctional fingers. These tasks require precise actions that only fingers can provide.

Although options for partial hand prostheses have increased greatly over the last decade, the abandonment of prosthetics remains relatively high. Some reasons for this abandonment include weight, difficulty of use, discomfort, lack of functionality, and unpredictability. Therefore, providing an improved prosthetic hand device that is lightweight, comfortable, and intuitive would greatly benefit amputees.

Strapping systems are commonly employed to secure partial hand prostheses to a user's anatomy. However, few improvements have been made to strapping systems, and little focus has been directed toward enhancing strapping components. Instead, the emphasis in partial hand prostheses often relates to the finger structures and methods for mechanical manipulation, while strapping systems are typically off-the-shelf products fabricated to incremental lengths for stock inventory, receiving minimal focus.

Current strap systems commonly feature aggressive hook and loop closures. While these materials facilitate donning and doffing with one hand, they lack durability and exhibit significant wear before failure. When an individual cannot use their opposite hand to don or doff the prosthetic device and instead relies on other means (e.g., mouth and teeth), hook and loop closures can be abrasive and uncomfortable. Hook and loop fasteners are also prone to frequently coming undone during normal use and being imprecise to adjust, complicating efforts to ensure a secure fit.

There is a need for an improved clasp mechanism for strapping and fastening applications to secure a partial hand prosthesis to a user. In particular, there is a need for a clasp mechanism that offers an adjustable, robust, and comfortable closure method while providing enhanced durability compared to existing strap systems used in prosthetic devices.

### SUMMARY

The present disclosure pertains to a clasp mechanism that connects a first strap segment to a second strap segment. Although the disclosure describes a beneficial application of the clasp mechanism within the context of a partial hand prosthesis, the embodiments and variations of the clasp mechanism are not restricted to prosthetic devices. Rather, they may be utilized in any appropriate device or application.

The clasp encompasses a cinch designed to receive a first strap segment, accompanied by a bracket affixed to the cinch that is connected to the second strap segment. The bracket is configured for removable attachment to the cinch via a channel situated between the strut and the second frame end. The cinch incorporates a strut that is positioned between and parallel to the first and second frame ends. The bracket features an aperture that exposes the free end of the second strap segment. Additionally, the cinch contains a strut laid out between and parallel to the first and second frame ends. This strut defines a compressive holder member that is intended to engage with the free end of the second strap segment through the bracket's aperture. The compressive holder member functions as a low load hold feature, delivering toggle force resistance through the compression of strap material.

Furthermore, the bracket delineates a tensile locking member that extends along the upper surface and is configured to engage with a projection formed on the second frame end of the cinch. The tensile locking member serves as a high load catch feature that secures the clasp mechanism during operation. Both the high-load catch feature and the low-load hold feature enhance the method for connecting and disconnecting the disclosed clasp mechanism.

The initial strap segment encircles a rod of the cinch, where the rod is aligned parallel to the strut and positioned between the first frame end and the strut of the cinch. The clasp mechanism may additionally comprise a retainer through which the first strap segment extends in an adjustable manner. This retainer is affixed to a distal end of the first strap segment and facilitates the translation of the first strap segment through lateral openings. Furthermore, the retainer secures the first strap segment, preventing it from retracting through the clasp and becoming disengaged.

In one embodiment, the cinch incorporates an upper tab, while the bracket consists of a lower tab positioned beneath and adjacent to the upper tab in a staggered arrangement. The staggered configuration of both the upper and lower tabs provides an enhanced quick-release solution suitable for one-handed operation. Moreover, this staggered design of the upper and lower tabs aids in donning and doffing without the use of hands (e.g., utilizing the mouth or teeth). The lower tab is pressed towards the second strap segment to disconnect the bracket from the cinch.

In another embodiment, the bracket is characterized by a major upper tab that extends over the rod and a part of the first strap segment when the bracket is firmly attached to the cinch. This major upper tab enhances the protection of the straps and components situated between the frame ends of the cinch. The major upper tab is lifted up and away from the cinch, compressing the strap material and allowing the tensile locking member to move beneath the protrusion, thereby disconnecting the bracket from the cinch.

In a further embodiment, the bracket features a major lower tab positioned between the second frame end and the second strap segment while the bracket is secured to the cinch. The major lower tab is pressed towards the second strap segment to disband the bracket from the cinch. By compressing the strap material and facilitating the movement of the tensile locking member beneath the protrusion, the bracket is enabled to slide through the channel and detach from the cinch.

The current disclosure pertains to a method for connecting a clasp. This method encompasses the following steps: (i) providing a first strap segment that is threaded through a cinch, where the cinch contains a strut positioned between and parallel to the first and second frame ends; (ii) inserting a bracket through a channel defined between the strut and the second frame end, with the bracket being connected to a second strap segment and featuring an aperture that reveals a free end of the second strap segment; (iii) engaging the free end of the second strap segment with a compressive holder member of the cinch via the aperture of the bracket; and (iv) compressing the free end of the second strap segment against the compressive holder member, as well as interlocking a tensile locking member of the bracket with a projection formed on the second frame end of the cinch.

The aforementioned features, aspects, and advantages of the present disclosure will be more clearly understood in the ensuing description, appended claims, and accompanying drawings.

### BRIEF DESCRIPTION OF THE DRAWINGS

The drawing figures are not necessarily to scale; rather, they are rendered to facilitate a clearer understanding of their components. These illustrations are not intended to be limiting in scope but rather to provide exemplary representations. The figures depict exemplary configurations of a clasp mechanism and do not constrain the structures or configurations expressed in the present disclosure.re.

FIG. 1 illustrates a perspective view of an exemplary partial hand prosthesis.

FIG. 2 illustrates a perspective view of an embodiment of the disclosed clasp mechanism.

FIG. 3 is a top view of the clasp mechanism in FIG. 2.

FIG. 4 is a side sectional view of the clasp mechanism in FIG. 3.

FIGS. 5A-5E depicts a clasping motion of the clasp mechanism in FIG. 4.

FIG. 6 illustrates a perspective view of an alternative embodiment of the clasp mechanism having a major upper tab configuration.

FIG. 7 is a side sectional view of the clasp mechanism in FIG. 6.

FIG. 8 illustrates a perspective view of an alternative embodiment of the clasp mechanism having a major lower tab configuration.

FIG. 9 is a perspective sectional view of the clasp mechanism in FIG. 8.

### DEFINITIONS

The term “clasp” refers to a device with interlocking parts, e.g., a cinch and a bracket, used for fastening things together.

The term “clasp mechanism” refers to the componentry of a clasp and corresponding strap segments and, optionally, retainer.

The terms “rigid,” “flexible,” and “resilient” may be used herein to distinguish characteristics of portions of certain features described in the present disclosure. The term “rigid” denotes that an element of the system generally lacks flexibility. On the other hand, the term “flexible” is intended to denote that features are capable of repeated bending. The features may be bent into retained shapes or do not retain a general shape, but continuously deform when force is applied. The term “resilient” qualifies such flexible features as returning to an initial general shape without permanent deformation. The term “semi-rigid” is used to connote properties of elements that provide support and are free-standing; however, such elements may have some degree of flexibility or resiliency.

The term “attached” refers to elements being connected or united by fastening, adhering, bonding, etc. by any method suitable for the elements being fastened, secured, or joined together with their constituent materials. Many suitable methods for attaching elements together are well-known, including adhesive bonding, pressure bonding, thermal bonding, mechanical fastening, etc. Such attachment methods may be used to attach elements over a particular area either continuously or intermittently. The term “attached” includes elements which are integrally formed with another element. The term “removably attached” refers to elements that may be connected or united, subsequently separated, and connected or united again by the same fastening means.

As used herein, the term “elastic” refers to any material which, upon application of a biasing force, is stretchable, that is, elongatable, at least about 60 percent (i.e., to a stretched, biased length, which is at least about 160 percent of its relaxed unbiased length), and which, will recover at least 55 percent of its elongation upon release of the stretching, elongation force. Some elastic materials may be elongated by more than 60 percent (i.e., much more than 160 percent of their relaxed length), for example, elongated 100 percent or more. Many of these materials will recover to substantially their initial relaxed length, for example, to within 105 percent of their initial relaxed length, upon release of the stretch force. Such materials are referred to herein as “highly elastic”. As used herein, the term “nonelastic” refers to any material that does not fall within the definition of “elastic” (or “elastomeric”) or “highly elastic” above.

### DETAILED DESCRIPTION OF VARIOUS EMBODIMENTS

Various embodiments disclosed herein relate to a clasp mechanism for securing two strap segments together. The clasp mechanism has particularly beneficial applications with prosthetic finger device fitted for a user with an amputated fingertip or finger segment. In preferred embodiments, the prosthetic finger is designed to articulate in a realistic, natural manner in response to movement in the user's residual finger and/or adjacent fingers.

Reference will now be made in detail to the accompanying drawings to facilitate an explanation of the clasp mechanism in applications, for example with a partial hand prosthesis discussed herein. The same reference numbers will be used throughout the drawings to refer to the same or like parts wherever possible. It is further understood that the use of relational terms such as first and second, top and bottom, and the like, if any, are used solely to distinguish one from another entity, item, or action without necessarily requiring or implying any actual such relationship or order between such entities, items, or actions.

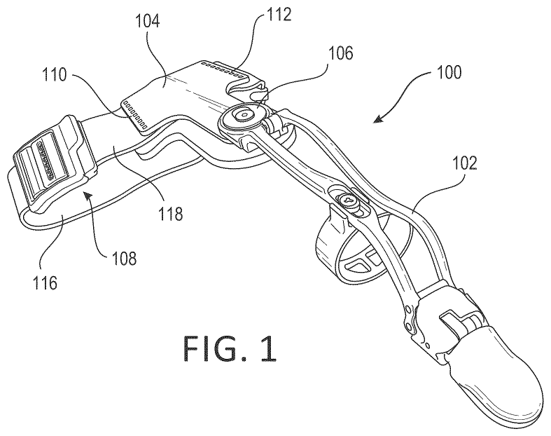

FIG. 1 illustrates a perspective view of an exemplary partial hand prosthesis 100 with a clasp mechanism 108 according to the disclosure. The partial hand prosthesis 100 comprises a prosthetic finger 102, e.g., as disclosed in U.S. Pat. No. 9,707,103, published on Jul. 18, 2017, the disclosure of which is incorporated by reference. In an embodiment, the prosthetic finger 102 includes a frame, articulation assembly, distal coupler, proximal rocker, hinged connection, and/or adjustable ring tendon. The embodiment depicted in FIG. 1 illustrates the prosthetic finger 102 as being connected to a backplate 104 by an eccentric metacarpophalangeal (MCP) pivot 106 configured for swivelable attachment to the hand of a user. The MCP pivot 106 comprises an articulation joint configured to rotate about an axis defined in the ventral-dorsal directions. Said articulation joint may be a pin, a screw, or any other appropriate fastener that joins the prosthetic finger 102 to the backplate 104. The clasp mechanism 108 of the partial hand prosthesis 100 connects first and second strap segments 116, 118 together. The first and second strap segments 116, 118 are anchored at first and second attachment portions 110, 112, respectively, of the backplate 104.

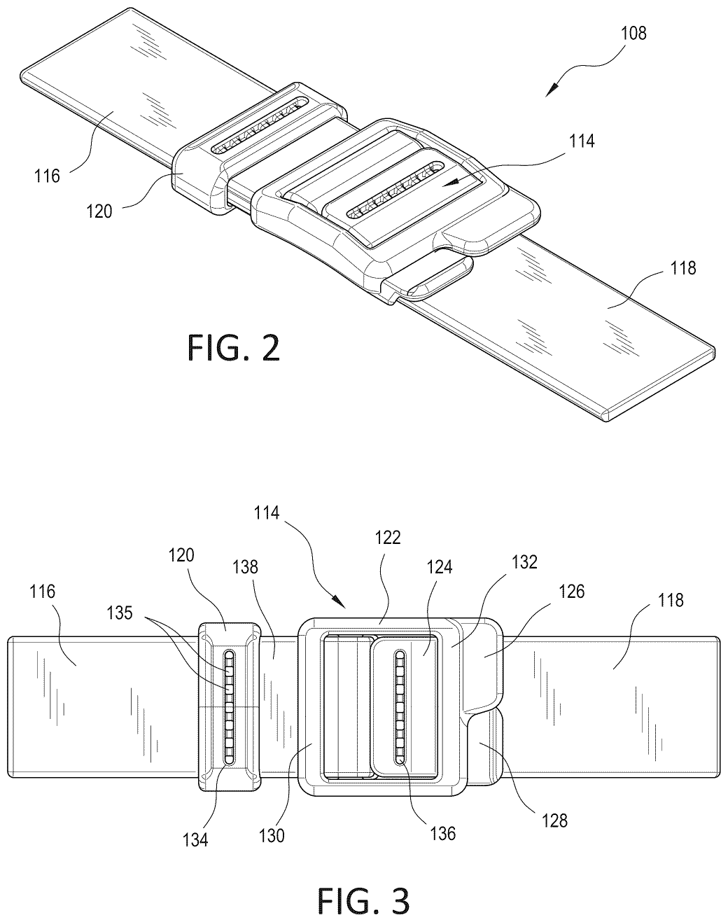

FIGS. 2-3 illustrate an embodiment of the clasp mechanism 108. In an embodiment, the clasp mechanism 108 is adapted to secure strap segments 116, 118 around a user's wrist and secure a proximal portion of the partial hand prosthesis 100 to the back of a user's hand. The clasp mechanism 108 comprises a cinch 122 and a bracket 124, which interlock and form a clasp 114 for connecting the first strap segment 116, attached to the first attachment portion 110, to the second strap segment 118, attached to the second attachment portion 112. The textile componentry of the strap segments 116, 118 is made of a deformable, hypoallergenic, non-absorbent, and mildew-resistant material. In a preferred embodiment, the strap segments 116, 118 are made of polyethylene terephthalate (PET) (e.g., Dacron); however, other deformable materials are also contemplated (e.g., nylon) for the construction of the strap segments 116, 118.

In an embodiment, the strap segments 116, 118 are inseparably fixed to the attachment portions 110, 112, respectively, of the backplate 104. In an alternative embodiment, the length of at least one strap segment 116 may be simplified by cutting a section of the strap segment 116 from a first end (i.e., the end arranged to be anchored at the first attachment portion 110). Following the permanent shortening of the at least one strap segment 116, the strap segment 116 is subsequently fixed to the backplate 104 at the attachment portion 110. This configuration increases modularity and customization of the clasp mechanism 108 with various strap systems. In an embodiment, the first and second strap segments 116, 118 are part of the same strap and define opposing portions at first and second ends of a continuous strap.

The clasp mechanism 108 comprises a clasp 114 for removably attaching the first strap segment 116 to the second strap segment 118. As noted above, existing strap systems for partial hand prosthesis include common failure points at hook and loop interfaces for donning and doffing. Using the novel clasp 114, the clasp mechanism 108 of the present disclosure avoids both (generally) hook and loop fasteners as the common donning and doffing approach and the reduced load-bearing suspension role of hook and loop fasteners.

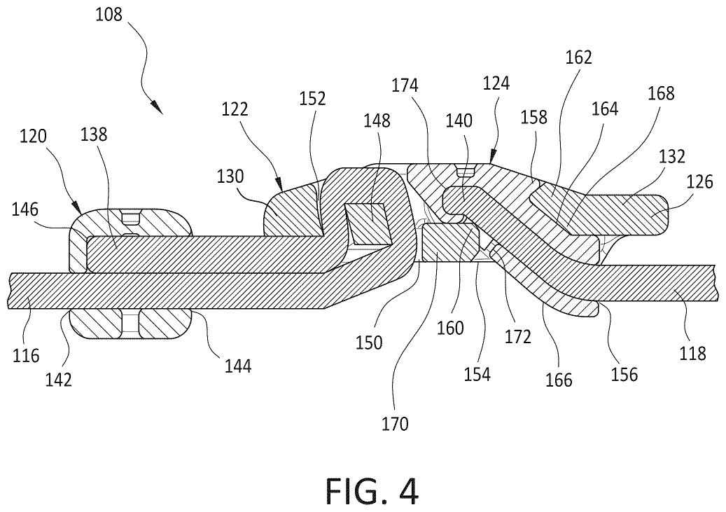

The clasp 114 comprises a cinch 122 and a bracket 124. The first strap segment 116 is adjustably connected to (i.e., threaded through) the cinch 122 and prevented from being disconnected from the cinch 122 by a retainer 120, as will be explained in greater detail below concerning FIG. 4. Extending from the first attachment portion 110, the first strap segment 116 passes through the retainer 120, wraps about a rod 148 of the cinch 122, and connects back to the retainer 120. The remote end 138 of the first strap segment 116 is retained within the retainer 120. The retainer 120 can adjust the length of the first strap segment 116 between the backplate 104 and the clasp 114 by sliding along first strap segment 116. In an embodiment, the remote end 138 of the first strap segment 116 may be attached to the retainer 120 via stitching channel 134. Stitching or threading material may pass through perforations 135 formed along the stitching channel 134.

The cinch 122 comprises a first frame end 130 proximate to the retainer 120 and an opposing second frame end 132. The second frame end 132 is arranged to engage with the bracket 124 to secure the first strap segment 116 and the second strap segment 118. The bracket 124 may also feature a stitching channel 136 for securing the second strap segment 118 to the bracket 124. In an embodiment, the clasp 114 features a staggered tab arrangement wherein the cinch 122 includes an upper tab 126 formed along the second frame end 132, and the bracket 124 includes a lower tab 128 beneath and adjacent to the upper tab 126. Such a configuration is advantageous for amputees donning and doffing the partial hand prosthesis 100 using only one hand.

FIG. 4 illustrates a detailed sectional view of the clasp mechanism 108. As observed, the first strap segment 116 extends into a first side opening 142 of the retainer 120 and exits through a second side opening 144. In connecting with the cinch 122, the first strap segment 116 extends through a first receptacle 150, formed between a rod 148 and strut 170 of the cinch 122, wraps around the rod 148 through a second receptacle 152, formed between the rod 148 and first frame end 130, wherein the remote end 138 of the first strap segment 116 returns to the retainer 120 through the second side opening 144. The remote end 138 is fixed within a recess 146 of the retainer 120. In an embodiment, the rod 148 has a polygon cross-sectional area having angled sides to define further the receptacles 150, 152 through which the first strap segment 116 extends to vary the degree of resistance against the first strap segment 116 and the cinch 122 for length adjustment.

The bracket 124 is configured and dimensioned to engage with the cinch 122 through a channel 154 of the cinch 122. The channel 154 is formed between the strut 170 and the second frame end 132. The free end 140 of the second strap segment 118 extends through a slot 156 of the bracket 124 to a distal cavity 174. The slot 156 is formed between an upper surface 164 and a lower surface 166 of the bracket 124. In an embodiment, when the clasp 114 is connected, the slot 156 entrance is substantially orthogonal to the upper tab 126 of the cinch 122. The bracket 124 includes an aperture 172 for partially exposing the free end 140 of the second strap segment 118. The strut 170 of the cinch 122 defines a compressive holder member 160 arranged for engaging with the free end 140 of the second strap segment 118 through the aperture 172 of the bracket 124. The compressive holder member 160 is a compressive, low load hold feature that provides toggle force resistance via strap material compression.

The bracket 124 includes a tensile locking member 158 formed along an upper surface 164, opposite the aperture 172. The tensile locking member 158 is configured and dimensioned to engage with a projection 162 formed along the second frame end 132 of the cinch 122. The tensile locking member 158 is a tensile, high load catch feature for securing the bracket 124 to the cinch 122 during wear. In an embodiment, when the bracket 124 is attached to the cinch 122, the upper surface 164 of the bracket 124 and a lower facet 168 of the cinch 122 correspond to one another and are aligned adjacent to the tensile locking member 158.

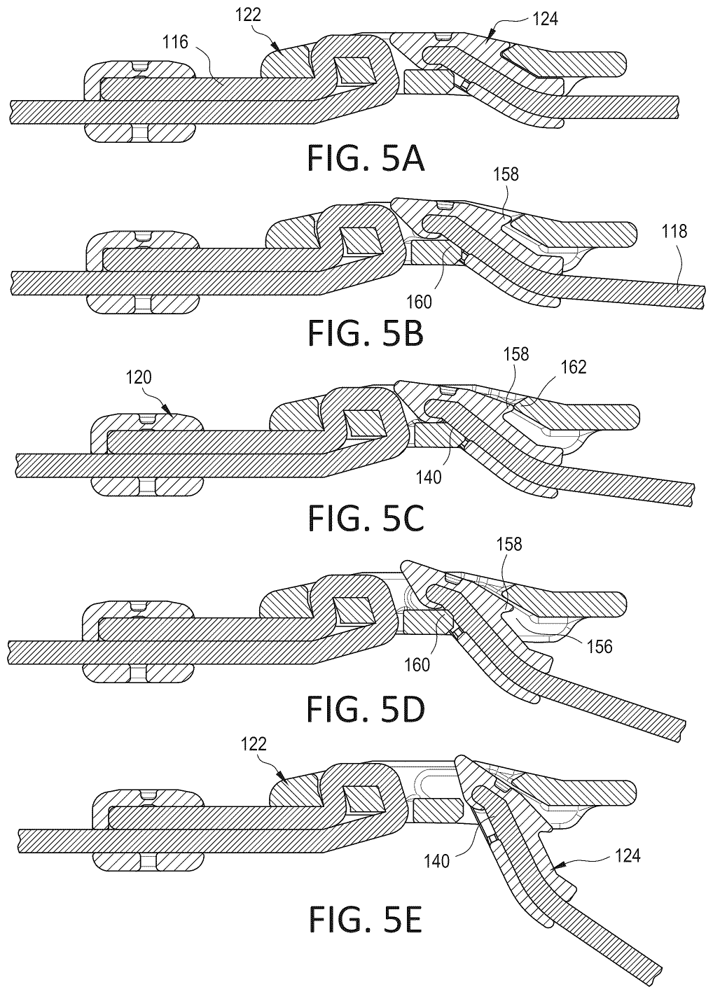

FIGS. 5A-5E illustrate the clasping (i.e., donning and doffing) motion of the clasp mechanism 108 depicted in FIG. 4. To initiate doffing, the lower tab 128 is pressed toward the second strap segment 118. In FIG. 5A, the bracket 124 is secured to the cinch 122, wherein the tensile locking member 158 is arranged to secure the partial hand prosthesis 100 to a user. Due to material properties of the bracket 124 and the cinch 122, a more rigid connection is established between the tensile locking member 158 and the projection 162 than the connection established with the compressive holder member 160 and the free end 140 of the second strap segment 118. The compressive holder member 160 exhibits greater compliance and flexibility for engagement between the bracket 124 and the cinch 122. Having both a high-load catch feature and low-load hold feature (i.e., tensile locking member 158 with the projection 162 and compressive holder member 160 with the free end 140) improves the method for both connecting and disconnecting the disclosed clasp mechanism 108, thereby improving the donning and doffing method for attaching a device (e.g., partial hand prosthesis 100) to a user. Moreover, the staggered tab design of the upper tab 126 and the lower tab 128 offers an improved quick-release solution for one-handed use. The staggered tab configuration of the upper tab 126 and the lower tab 128 further allows for donning and doffing without hands (i.e., using the mouth or teeth).

In FIG. 5B, as the tensile locking member 158 slides against the projection 162 of the cinch 122, the free end 140 of the second strap segment 118 is compressed against the compressive holder member 160. FIG. 5C illustrates the free end 140 under maximum compression against the compressive holder member 160 as the fringes of the tensile locking member 158 and the projection 162 interact. In FIG. 5D, after the tensile locking member 158 disengages with the projection 162 and enters into the channel 154, compressive forces against the free end 140 diminish to permit doffing. FIG. 5E illustrates the bracket 124 sliding through the channel 154 after the compressive holder member 160 disengages with the free end 140 to subsequently permit doffing. The reverse order of FIGS. 5A-5E illustrates the steps for linking the clasp mechanism 108.

The present disclosure relates to a method for connecting the clasp and comprises providing a first strap segment 116 and a cinch 122, the first strap segment 116 being threaded through the cinch 122, wherein the cinch 122 includes a strut 170 arranged between and parallel to first and second frame ends 130, 132, and inserting a bracket 124 through a channel 154 defined between the strut 170 and the second frame end 132 of the cinch 122, wherein the bracket 124 is connected to a second strap segment 118 and includes an aperture 172 that exposes a free end 140 of the second strap segment 118. The method includes engaging the free end 140 of the second strap segment 118 with a compressive holder member 160 of the cinch 122 through the aperture 172 of the bracket 124. The connection between the compressive holder member 160 and the free end 140 provides a compressive, low load hold feature with toggle force resistance via material compression of the second strap segment 118. After compressing the free end 140 of the second strap segment 118 against the compressive holder member 160, a tensile locking member 158 of the bracket 124 is interlocked with a projection 162 formed on the second frame end 132 of the cinch 122.

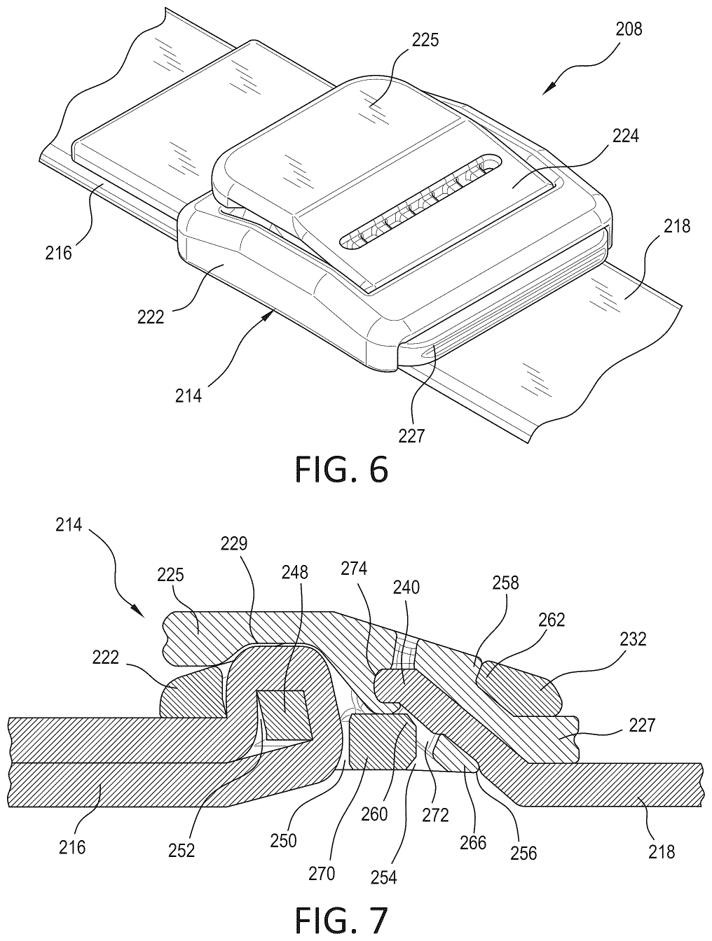

FIGS. 6-7 illustrate an alternative embodiment of the clasp mechanism 208 having a clasp 214 with a major upper tab 225. The first strap segment 216 extends through a first receptacle 250 of the cinch 222 and about a rod 248 of the cinch 222. The rod 248 is formed between first and second receptacles 250, 252 configured and dimensioned to receive the first strap segment 216. When fully clasped, the portion of the first strap segment 216 extending about the rod 248 is partially covered within a groove 229 of the major upper tab 225. As depicted, the major upper tab 225 extends over the first frame end 230, whereas a minor lower tab 227 extends below second frame end 232.

The bracket 224 is configured to interface with the cinch 222 through a channel 254 of the cinch 222. The channel 254 is formed between the strut 270 and the second frame end 232. The free end 240 of the second strap segment 218 extends through a slot 256 of the bracket 224 to a distal cavity 274. In an embodiment, when the clasp 214 is connected, the slot 256 entrance is substantially parallel to the minor lower tab 227 of the bracket 224. The bracket 224 includes an aperture 272 for partially exposing the free end 240 to engage with a compressive holder member 260 defined by the strut 270 of the cinch 222. The aperture 272 is formed along a lower surface 266 of the bracket 224. The compressive holder member 260 is a low load hold feature that provides toggle force resistance via strap material compression. The bracket 224 includes a tensile locking member 258 configured and dimensioned to engage with a projection 262 formed along the second frame end 232 of the cinch 222. The tensile locking member 258 is a tensile, high load catch feature for securing the bracket 224 to the cinch 222 during wear. The major upper tab 225 is pulled up and away from the cinch 222 so that the bracket 224 rotates about the strut 270 to initiate doffing or disconnection of the clasp 214.

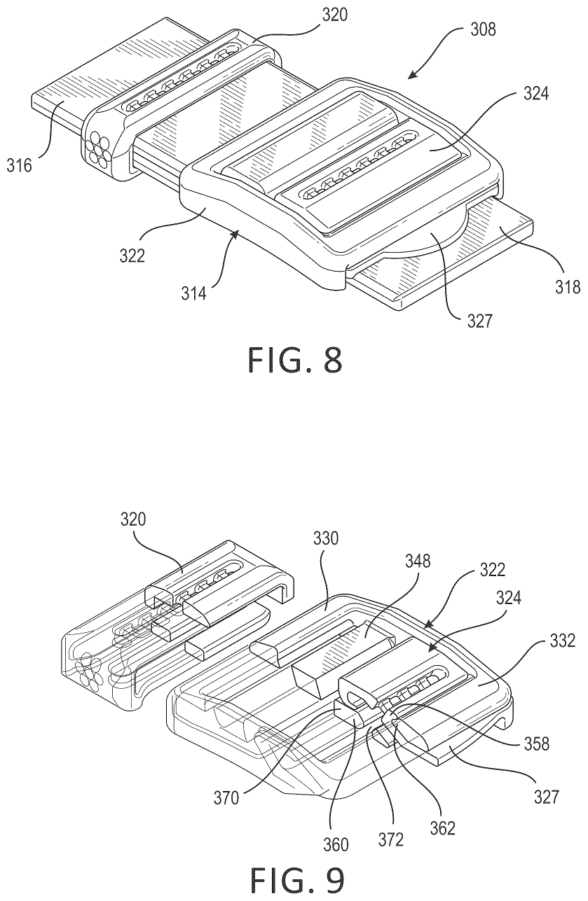

FIGS. 8-9 illustrate an alternative embodiment of the clasp mechanism 308 having a clasp 314 with a major lower tab 327. The clasp 314 comprises a cinch 322 and a bracket 324. The first strap segment 316 is adjustably connected to (i.e., threaded through) the cinch 322 and prevented from being disconnected from the cinch 322 by a retainer 320. The first strap segment 316 passes through the retainer 320, wraps about a rod 348 of the cinch 322, extends below the first frame end 330, and connects back to the retainer 320. The retainer 320 can adjust the first strap segment 316 length between the backplate (e.g., backplate 104) and the clasp 314.

The bracket 324 is configured to interface with the cinch 322 between the rod 348 and the second frame end 332. The bracket 324 includes an aperture 372 for partially exposing the second strap segment 318 to engage with a compressive holder member 360 defined by the strut 370 of the cinch 322. The compressive holder member 360 is a low load hold feature that provides toggle force resistance via strap material compression. The bracket 324 further includes a tensile locking member 358 configured to engage with a projection 362 formed along the second frame end 332 of the cinch 322. The tensile locking member 358 is a tensile, high load catch feature for securing the bracket 324 to the cinch 322 during wear. The major lower tab 327 of the bracket 324 is pressed toward the second strap segment 318 to initiate doffing or disconnection of the clasp 314.

It is acknowledged that not all objects or advantages may be attained under any embodiment of the disclosure. Individuals skilled in the art will recognize that the disclosed clasp mechanism may be implemented or executed in a manner that achieves or optimizes one advantage or group of advantages as elucidated herein without necessarily achieving other objects or advantages as articulated or suggested herein.

The proficient artisan shall recognize the interchangeability of various disclosed features. In addition to the variations described herein, other known equivalents for each feature may be combined and employed by one of ordinary skill in this field to develop and utilize a clasp mechanism in accordance with the principles of the current disclosure. Consequently, this disclosure, along with its embodiments and variations, is not confined to prosthetic devices but can be applied to any appropriate device.

Although this disclosure delineates certain exemplary embodiments and instances of a prosthetic device, it shall be understood by those skilled in the art that the present disclosure extends beyond the specified embodiments to include other alternative embodiments and/or applications of the disclosure, as well as apparent modifications and equivalents. It is intended that the scope of the present disclosure be not restricted by the disclosed embodiments as described above, with the disclosed clasp mechanism applicable in other domains, including but not limited to belts, restraints, handles, luggage, girdles, bands, harnesses, and similar constructs.

The invention claimed is:

1. A clasp mechanism comprising:
a cinch arranged to receive a first strap segment; and
a bracket for removably attaching to the cinch, the bracket being connected to a second strap segment;
wherein the bracket includes an aperture exposing a free end of the second strap segment;
wherein the cinch includes a strut arranged between and parallel to first and second frame ends;
wherein the strut of the cinch defines a compressive holder member arranged for engaging with the free end of the second strap segment through the aperture of the bracket.

2. The clasp mechanism of claim 1, wherein the bracket defines a tensile locking member formed along an upper surface and configured to engage with a projection formed on the second frame end of the cinch.

3. The clasp mechanism of claim 1, wherein the bracket is arranged to removably attach to the cinch through a channel defined between the strut and the second frame end.

4. The clasp mechanism of claim 1, wherein the first strap segment wraps about a rod of the cinch, the rod being parallel to the strut and arranged between the first frame end and the strut of the cinch.

5. The clasp mechanism of claim 4, wherein the bracket defines a major upper tab arranged to extend over the rod and a portion of the first strap segment when the bracket is secured to the cinch.

6. The clasp mechanism of claim 5, wherein the major upper tab is pulled up and away from the cinch to disconnect the bracket from the cinch.

7. The clasp mechanism of claim 1, further comprising a retainer through which the first strap segment adjustably extends;
wherein the retainer is attached to a remote end of the first strap segment and permits translation of the first strap segment through side openings.

8. The clasp mechanism of claim 1, wherein the cinch includes an upper tab, and the bracket includes a lower tab arranged below and adjacent to the upper tab in a staggered configuration.

9. The clasp mechanism of claim 8, wherein the lower tab is pressed toward the second strap segment to disconnect the bracket from the cinch.

10. The clasp mechanism of claim 1, wherein the bracket defines a major lower tab arranged between the second frame end and the second strap segment when the bracket is attached to the cinch;
wherein the major lower tab is pressed toward the second strap segment to disconnect the bracket from the cinch.

11. A method for connecting a clasp, the method comprising:
providing a first strap segment connected to a cinch of the clasp, the cinch including a strut arranged between and parallel to first and second frame ends;
inserting a bracket of the clasp through a channel defined between the strut and the second frame end, the bracket being connected to a second strap segment and including an aperture exposing a free end of the second strap segment;
engaging the free end of the second strap segment with a compressive holder member of the cinch through the aperture of the bracket;
compressing the free end of the second strap segment against the compressive holder member; and
interlocking a tensile locking member of the bracket with a projection formed on the second frame end of the cinch.

12. The method of claim 11, further comprising adjustably extending the first strap segment through a retainer;
wherein the retainer is attached to a remote end of the first strap segment and permits translation of the first strap segment through side openings.

13. The method of claim 11, wherein the cinch includes an upper tab, and the bracket includes a lower tab arranged below and adjacent to the upper tab in a staggered configuration.

14. The method of claim 13, further comprising pressing the lower tab toward the second strap segment to disconnect the bracket from the cinch.

15. The method of claim 11, wherein the first strap segment wraps about a rod of the cinch, the rod being parallel to the strut and arranged between the first frame end and the strut of the cinch.

16. The method of claim 15, wherein the bracket defines a major upper tab arranged to extend over the rod and a portion of the first strap segment when the bracket is secured to the cinch.

17. The method of claim 11, wherein the bracket defines a major lower tab arranged between the second frame end and the second strap segment when the bracket is attached to the cinch;
wherein the major lower tab is pressed toward the second strap segment to disconnect the bracket from the cinch.

18. A clasp mechanism comprising:
a clasp for removably attaching a first strap segment to a second strap segment;
wherein a free end of the second strap segment is housed within a bracket of the clasp;
wherein the bracket is configured to interlock with a cinch of the clasp;
wherein the bracket includes an aperture exposing the free end of the second strap segment;
wherein a strut of the cinch defines a compressive holder member arranged for engaging with the free end of the second strap segment through the aperture of the bracket;
wherein the first strap segment is adjustably connected to the cinch of the clasp;
wherein the bracket defines a tensile locking member formed along an upper surface of and the bracket, the tensile locking member being configured and dimensioned to engage with a projection formed by the cinch.

19. The clasp mechanism of claim 18, wherein the first strap segment wraps about a rod of the cinch, the rod being parallel to the strut and arranged between the first frame end and the strut of the cinch;
wherein the bracket defines a major upper tab arranged to extend over the rod and a portion of the first strap segment when the bracket is secured to the cinch.

20. The clasp mechanism of claim 18, wherein the bracket is arranged to removably attach to the cinch through a channel defined between the strut and the second frame end.
